(19) United States
(12) Patent Application Publication
(10) Pub. No.: US 2003/0039620 A1
(43) Pub. Date: Feb. 27, 2003

(54) Transfer resistant, non-tacky, liquid cosmetic compositions for covering skin discolorations and imperfections

(76) Inventors: Victor Ruben Rodriguez, Fairfield, OH (US); Karl Shiqing Wei, Mason, OH (US); Mannie Lee Clapp, Mason, OH (US); Mark David Evans, Cincinnati, OH (US); Carla Jean Ritchie, Hamilton, OH (US); Edward Dewey Smith III, Mason, OH (US); Rebecca Ann Taylor, Fairfield, OH (US); Cheyne Pohlman Thomas, Highland Heights, KY (US); Peter Jonathan Wyatt, Belair, MD (US); Jorge Max Sunkel, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/949,953

(22) Filed: Sep. 10, 2001

Related U.S. Application Data
(63) Continuation of application No. 09/849,113, filed on May 4, 2001, now abandoned.

(51) Int. Cl. A61K 7/021

(52) U.S. Cl. 424/63

(57) ABSTRACT

Disclosed are liquid cosmetic compositions comprising an organosiloxane resin; a fluid diorganopolysiloxane polymer; a volatile carrier liquid; from about 15% to about 50% by weight of a solid pigment such that the weight ratio of the fluid diorganopolysiloxane polymer and organosiloxane resin to the solid pigment is from about 0.50 to about 2.5; and from about 0.5% to about 15% by weight of an inorganic thickening solid or from about 0.1% to about 20% by weight of a silicone elastomer; wherein the weight ratio of the organosiloxane resin to the fluid diorganopolysiloxane polymer is from about 1:2 to about 10:1. Also disclosed are liquid cosmetic compositions characterized by an average Sweat Wear Index of greater than about 60%, an average Coverage Index of greater than about 80%, and an average Tackiness Index of less than 25%. The compositions are applied topically to cover skin imperfections and discolorations, especially those associated with varicose and spider veins, to provide improved wear resistant coverage.

### FIELD OF INVENTION

The present invention relates to transfer resistant, non-tacky, liquid cosmetic compositions which preferably contain an organosiloxane resin, a fluid diorganopolysiloxane polymer, a volatile carrier, and a pigment selected for transfer resistant characteristics. The present invention also relates to methods of applying the compositions to cover or hide skin discolorations and imperfections, including varicose and spider veins.

### BACKGROUND OF THE INVENTION

Cosmetic compositions are known for application to and coverage of skin imperfections and discolorations, including cosmetics directed specifically to covering or hiding varicose or spider veins. These cosmetic products are typically liquid or soft solid formulations that contain a suitable pigment, a volatile liquid carrier, a suspending agent for the pigment, and a non-volatile material to provide substantivity for the pigment during and after application.

Many of the cosmetic compositions known for topical application to cover skin imperfections and discolorations provide good coverage to the applied area over short periods of time, but tend to wear away shortly after application, especially when applied to those areas of the skin in contact with clothing. These types of cosmetic compositions often require reapplication after only a few hours to thus maintain the desired degree of coverage.

Responsive to the need for extended wear cosmetics, the cosmetic art has since described a number of extended wear technologies directed to the application of cosmetics to the desired area of the skin which do not require frequent reapplication to maintain cosmetic coverage. Among the extended wear cosmetics described in the literature include those that contain a combination of solid pigments, organosiloxane resins, fluid diorganopolysiloxane polymers, and a volatile carrier. It has been found that these compositions provide excellent extended wear performance when applied to the lips or other desired area of the skin. Although these extended wear compositions provide excellent extended wear performance, they tend to be tacky or sticky during and after product application and do not always provide optimal coverage from a liquid formulation base when the composition is applied over relatively large surface areas such as those areas of the legs affected by varicose or spider veins.

It has now been found that extended wear cosmetics can be formulated to deliver a smooth non-tacky feel with good coverage over relatively large surface areas, provided that the formulation has a defined set of functional characteristics as defined herein, or otherwise contains an organosiloxane resin; a fluid diorganopolysiloxane polymer; a volatile carrier liquid; from about 15% to about 50% by weight of a solid pigment such that the weight ratio of the fluid diorganopolysiloxane polymer and organosiloxane resin to the solid pigment is from about 0.50 to about 2.5; and inorganic thickening solids or gels contains those solids such as Bentone® Gel ISD or other similar materials that provide from about 0.5% to about 15% of an inorganic thickening solid by weight of the formulation; wherein the weight ratio of the organosiloxane resin to the fluid diorganopolysiloxane polymer is from about 1:2 to about 10:1. Especially important to provide the desired non-tacky skin feel and optimal coverage is 1) the defined weight ratio of the resin to fluid polymer, 2) a solid pigment concentration of at least 15% by weight of the composition, and 3) selection of an inorganic thickening solid as the suspending or thickening agent.

It is therefore an object of the present invention to provide an extended wear cosmetic composition, and corresponding methods of application, suitable for non-tacky application to or coverage over large areas of the skin, especially to those areas having skin imperfections or discolorations such as spider or leg veins. It is a further object of the present invention to provide such an extended wear cosmetic compositions, and corresponding methods of application, that provide improved coverage over relatively large areas of the skin.

### SUMMARY OF THE INVENTION

The present invention relates to liquid cosmetic compositions and corresponding methods of application for covering or hiding skin imperfections or discolorations. These compositions and methods are directed to or comprise an organosiloxane resin; a fluid diorganopolysiloxane polymer; a volatile carrier liquid; from about 15% to about 50% by weight of a solid pigment such that the weight ratio of the fluid diorganopolysiloxane polymer and organosiloxane resin to the solid pigment is from about 0.50 to about 2.5; and inorganic thickening solids or gels contains those solids such as Bentone® Gel ISD or other similar materials that provide from about 0.5% to about 15% of an inorganic thickening solid by weight of the formulation; wherein the weight ratio of the organosiloxane resin to the fluid diorganopolysiloxane polymer is from about 1:2 to about 10:1. The compositions can also be defined by functional characteristics as defined herein, which includes a average Coverage Index of at least about 80%, an average Sweat Wear Index of at least about 60%, and an average Tackiness Index of less than about 25%.

It has been found that the compositions and methods of the present invention provide improved wear-resistant coverage over skin imperfections and discolorations. It has been found that such wear resistant performance is preferably made possible by combining a defined ratio of organosiloxane resin and fluid diorganopolysiloxane polymer, along with an inorganic thickening solid and a relatively high pigment solids concentration of at least about 15% by weight of the composition. The compositions are applied topically to cover skin imperfections and discolorations, especially those associated with varicose and spider veins, to provide improved wear resistant coverage especially on those areas of the skin that come in direct contact with clothing. Unlike other wear-resistant cosmetics, these compositions provide a smooth, non-tacky skin feel during and after application to the desired area of the skin.

It has been found that the compositions and methods of the present invention can be further improved for skin feel performance by adding or formulating with a silicone elastomer in the form of a powder or gel in the cosmetic compositions of the present invention, while also maintaining the desired functional indices as defined herein.

### BRIEF DESCRIPTION OF THE FIGURES

FIG. 1 is a planar view of an apparatus or weight as referenced hereinafter in the test methodologies directed to determining Dry Wear Index, Sweat Wear Index, and Oil Wear Index values for the liquid cosmetic compositions of the present invention.

FIG. 2 is a planar view of an apparatus as referenced hereinafter in the test methodology directed to determining Film Flexibility values for the liquid cosmetic compositions of the present invention.

### DETAILED DESCRIPTION

The cosmetic compositions of the present invention preferably comprise a select combination of an organosiloxane resin, a fluid diorganopolysiloxane polymer, a volatile carrier, an inorganic thickening system and a solid cosmetic pigment. These and other essential limitations of the compositions and methods of the present invention, as well as many of the optional ingredients suitable for use herein, are described in detail hereinafter.

The term “anhydrous” as used herein, unless otherwise specified, refers to those compositions or materials containing less than about 10%, more preferably less than about 5%, even more preferably less than about 3%, even more preferably zero percent, by weight of water. The cosmetic compositions of the present invention are preferably anhydrous.

The term “volatile” as used herein, unless otherwise specified, refers to those materials having an average boiling point at one (1) atmosphere of pressure (atm) of less than about 250° C., more typically less than about 235° C. at one (1) atm.

The term “ambient conditions” as used herein refers to surrounding conditions at one atmosphere of pressure, 50% relative humidity, and 25° C.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

The cosmetic compositions and methods of the present invention can comprise, consist of, or consist essentially of the essential elements and limitations of the invention described herein, as well as any additional or optional ingredients, components, or limitations described herein or otherwise useful in personal care compositions intended for topical application to the skin.

### Organosiloxane Resins

The liquid cosmetic compositions of the present invention may comprise an organosiloxane resin in combination with a fluid diorganopolysiloxane polymer as described in detail hereinafter. The organosiloxane resin suitable for use herein can be any organosiloxane resin that can be solubilized within the composition and that is otherwise compatible with the other essential ingredients of the composition.

It has been found that the weight ratio of the organosiloxane resin to the fluid diorganopolysiloxane polymer in the liquid cosmetic compositions of the present invention range from about 1:2 to about 10:1, more preferably from about 1:1 to about 5:1, even more preferably from about 1:1 to about 3:1, wherein the total concentration of the organosiloxane resin/fluid diorganopolysiloxane polymer combination preferably ranges from about 10% to about 40%, more preferably from about 15% to about 30%, even more preferably from about 20% to about 30%, by weight of the composition.

The organosiloxane resin for use in the liquid cosmetic compositions of the present invention includes combinations of R₃SiO_{1/2} (M units), R₂SiO (D units), RSiO_{3/2} (T units), SiO₂ (Q units) in ratios to each other that satisfy the relationship RₙSiO(_{4-n)/2} where n is a value between 1.0 and 1.50 and R is a methyl group. Note that a small amount, up to 5%, of silanol or alkoxy functionality may also be present in the resin structure as a result of processing. The organosiloxane resins are solids at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier.

Preferred organosiloxane resins for use in the liquid cosmetic compositions are those that contain repeating monofunctional or R₃SiO_{l/2} (M units) and quadrafunctional or SiO₂ (Q units), otherwise known as “MQ” resins as described in U.S. Pat. No. 5,330,747 (Krzysik), which descriptions are incorporated herein by reference. Examples of highly preferred organosiloxane resins are those in which the ratio of “M” to “Q” functional units is about 0.5 and the value of n is 1.5, non limiting examples of which are commercially available from Wacker Silicones Corporation of Adrian Mich. (e.g., Wacker 803 and 804) and the General Electric Company (e.g., G. E. 1170-002).

Other suitable organosiloxane resins includes functionalized silicone resins such as silicone ester waxes comprising moieties of the general formula:

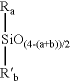

wherein R is an organic radical, R′ is a carboxylic acid ester, “a” and “b” are integers independently either 1 or 2 wherein a+b equals 2 or 3.

It has been found that the methods of the present invention can also be directed to and include the application of any topical composition containing an organosiloxane resin as defined herein to those areas of the skin discolored by visible varicose veins, spider veins, or combinations thereof. Such compositions preferably provide the functional product characteristics as defined herein, and preferably further comprise other ingredients as defined herein such as a volatile carrier liquid, a fluid diorganopolysiloxane polymer, a solid pigment, and a suitable thickening agent such as an inorganic thickening solid or silicone elastomer.

### Fluid Diorganopolysiloxane Polymers

The liquid cosmetic compositions of the present invention may also comprise a fluid diorganopolysiloxane polymer in combination with the organosiloxane resin described hereinbefore. The fluid diorganopolysiloxane polymer suitable for use herein can be any fluid diorganopolysiloxane polymer material that can be solubilized within the composition and that is otherwise compatible with the other essential ingredients of the composition.

The concentration of the fluid diorganopolysiloxane polymer in the liquid cosmetic composition of the present invention depends upon the total resin/fluid polymer concentration and relative weight ratios as described hereinbefore, but will generally range from about 3% to about 15%, more typically from about 5% to about 10%, even more typically from about 5% to about 8%, by weight of the composition.

The fluid diorganopolysiloxane polymers for use in the liquid cosmetic compositions herein have a preferred viscosity of from about 100,000 to about 25,000,000 centistokes (cSt) at 25° C. and are readily soluble in the volatile carrier described hereinafter and therefore preferably form a single phase solution when combined with both the volatile carrier and the organosiloxane resin.

The fluid diorganopolysiloxane polymers for use in the liquid cosmetic compositions comprise repeating units that correspond to the formula (R₂SiO), where R is a monovalent hydrocarbon radical containing from 1 to 6 carbon atoms, preferably R is selected from methyl, ethyl, propyl, isopropyl, butyl, isobutyl, t-butyl, amyl, hexyl, vinyl, allyl, cyclohexyl, phenyl, fluoroalkyl and mixtures thereof. The fluid diorganopolysiloxane polymers may contain one or more of these hydrocarbon radicals as substituents on the siloxane polymer backbone. The fluid diorganopolysiloxane polymers may be terminated by triorganosilyl groups of the formula (R′₃Si) where R′ is a radical selected from monovalent hydrocarbons containing from 1-6 carbon atoms, hydroxyl groups, alkoxyl groups and mixtures thereof.

Non limiting examples of preferred fluid diorganopolysiloxane fluids for use herein include poly(dimethylsiloxane) [PDMS] materials such as those available from General Electric as SE30, SE72, SE84, Viscasil®100M, and Baysilone Fluid M 500,000.

### Volatile Carrier

The liquid cosmetic compositions of the present invention may also comprise a volatile liquid carrier suitable for topical application to the skin that is also compatible with the essential materials selected for use herein. The volatile liquid carrier is typically a liquid under ambient conditions or otherwise in liquid form as formulated within the compositions, and solubilizes the fluid diorganopolysiloxane polymer/organosiloxane resin combination within the composition or otherwise helps to maintain the combination as solubilized within the composition.

The volatile liquid carrier for use in the liquid cosmetic compositions of the present invention include volatile hydrocarbons, volatile silicones and combinations thereof, the concentration of which within the liquid cosmetic compositions generally ranges from about 10% to about 90%, preferably from about 20% to about 80%, and more preferably from about 40% to about 60%, by weight of the compositions.

Volatile hydrocarbons suitable for use as a volatile liquid carrier in the liquid cosmetic compositions include those hydrocarbons having boiling points in the range of from about 60° C. to about 260° C., more preferably volatile hydrocarbons having from about C₈ to about C₂₀ chain lengths, more preferably C₈ to C₂₀ isoparaffins. Preferred isoparaffins for use herein include isododecane, isohexadecane, isoeocosane, 2,2,4-trimethylpentane, 2,3-dimethylhexane and combinations thereof. Most preferred is isododecane.

Volatile silicones suitable for use as a volatile liquid carrier in the liquid cosmetic compositions herein include those volatile silicones as described in Todd et al., “Volatile Silicone Fluids for Cosmetics”, Cosmetics and Toiletries, 91:27-32 (1976), which descriptions are incorporated herein by reference. The volatile silicone can be linear, cyclic or branched, but is preferably a cyclic silicone having from about 3 to about 7, more preferably from about 4 to about 5, silicon atoms. Most preferably are those which conform to the formula:

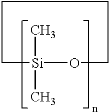

wherein n is from about 3 to about 7, preferably from about 4 to about 5, most preferably 5. Suitable volatile silicones for use herein include, but are not limited to, Cyclomethicone D-5 (commercially available from G. E. Silicones); Dow Corning 344, and Dow Corning 345 (commercially available from Dow Corning Corp.); GE 7207, GE 7158 and Silicone Fluids SF-1202 and SF-1173 (available from General Electric Co.); SWS-03314, SWS-03400, F-222, F-223, F-250, F-251 (available from SWS Silicones Corp.); Volatile Silicones 7158, 7207, 7349 (available from Union Carbide); Masil SF-V ( available from Mazer) and combinations thereof. Cyclopentasiloxane is most preferred among the volatile silicone liquids.

### Solid Pigment

The liquid cosmetic compositions of the present invention may also comprise solid pigment particles at concentrations ranging from a minimum of at least about 15% by weight of the composition. Preferred concentrations range from about 15% to about 30%, more preferably from about 15% to about 25%, by weight of the composition. The solid pigment is also be formulated within the liquid cosmetic compositions so that the weight ratio of the fluid diorganopolysiloxane polymer/organosiloxane resin combination to the solid pigment is from about 0.50 to about 2.5, preferably from about 0.5 to about 1.5.

The solid pigment includes any solid organic or inorganic color or pigment suitable for topical application to the skin that is also insoluble in the selected product matrix or otherwise remains at least partially in the form of dispersed or suspended solids within the matrix.

The solid pigment suitable for use in the liquid cosmetic compositions is preferably a titanium oxide, iron oxide, chalk, talc, iron oxides, titanated micas, and combinations thereof. More preferred are titanium oxide, iron oxide, and combinations thereof.

### Inorganic Thickening System

The liquid cosmetic compositions of the present invention may also comprise an inorganic thickening system that contains an inorganic thickening solid, wherein the concentration of the inorganic thickening solid in the compositions ranges from about 0.5% to about 15%, more preferably from about 1.0% to about 5.0%, even more preferably from about 2.0% to about 4.0%, by weight of the compositions.

The inorganic thickening system may be formulated into the composition in the form of finely divided inorganic solids, or preferably in the form of a gel that had previously been formed from a combination of the inorganic solids with a suitable liquid carrier. Inorganic solids suitable for use in the inorganic thickening system include bentonite or other similar clay materials, and also including finely divided silica, including fumed silicas (e.g., Cab-O-Sil).

The inorganic thickening solids are preferably those derived from or otherwise similar to inorganic clays, preferably montmorillonite clays. Montmorillonite clays are those which contain the mineral montmorillonite and are characterized by having a suspending lattice. Examples of montmorillonite clays suitable for use herein include the bentonites, hectorites, colloidal magnesium aluminum silicates, and mixtures thereof.

Highly preferred inorganic thickening solids include hydrophobically treated montmorillonite clays, including hydrophobic bentonites available from Rheox, Inc., Hightstown, N.J., U.S.A., under the tradename Bentone®. Non limiting examples of suitable Bentone® materials for use herein include Bentone® 27, Bentone® 34, Bentone® 38, Bentone® EW, Bentone® Gel CAO, Bentone® Gel IPM Bentone® Gel ISD, Bentone® Gel LOI, Bentone® Gel MIO, Bentone® Gel MIO, Bentone® Gel MIO A-40, and many other similar materials. Most preferred are Bentone® Gels that contain hydrophobically treated bentonite clays in the form of quaternium-18 hectorite, a non limiting example of which is Bentone Gel ISD which is a gel material containing 10% quatemium-18 hectorite, 87% isododecane and 3% propylene carbonate.

As described in greater detail hereinafter, it has also been found that silicone elastomers can be used in addition to or in place of the inorganic thickening solids.

### Silicone Elastomer

The liquid cosmetic compositions of the present invention may further comprise a silicone elastomer suitable for topical application to the skin, which is used in addition to or in place of the inorganic thickening solids as described hereinbefore. The silicone elastomer is incorporated into the liquid cosmetic compositions in the form of a silicone gel or silicone powder material, wherein the silicone gel or powder most typically comprises a volatile silicone liquid in combination with the silicone elastomer. Silicone elastomer concentrations in the liquid cosmetic compositions of the present invention preferably range from about 0.1% to about 20%, more preferably from about 0.5% to about 10%, even more preferably from about 1% to about 5%, by weight of the composition

Silicone elastomers suitable for use in the liquid cosmetic compositions of the present invention include any silicone elastomer that is known or otherwise suitable for topical application to the skin. The term “silicone elastomer” as used herein refers to the art-recognized definition of silicone elastomers, which in its broadest reading includes any chemically crosslinked siloxane polymer. No specific restriction exists as to the type of organopolysiloxane that can serve as a starting material for preparing the crosslinked organopolysiloxane (silicone elastomer).

The silicone elastomer for use in the liquid cosmetic compositions of the present invention may be emulsifying, non-emulsifying or combinations thereof. The term “non-emulsifying,” as used herein refers to those crosslinked organopolysiloxanes from which oxyalkylene units are absent. Non-limiting examples of non-emulsifying silicone elastomers suitable for use in the liquid cosmetic compositions of the present invention are described in U.S. Pat. No. 6,103,250 (Edwards et al., issued Aug. 24, 1999) and U.S. Pat. No. 5,922,308 (Brewster et al., issued Jul. 13, 1999), which descriptions are incorporated herein by reference. Non-limiting examples of a specific silicone gel material containing a non-emulsifying elastomer for use herein are the cyclomethicone (and) dimethicone crosspolymer available from Dow Corning under the tradename DC-9040, and SFE-168 and SFE-839 available from GE Silicones.

The term “emulsifying,” as used herein, refers to those crosslinked organopolysiloxanes having at least one oxyalkylene (e.g., polyoxyethylene or polyoxypropylene) unit. Preferred emulsifying elastomers for use in the cosmetic compositions of the present invention include polyoxyalkylene-modified elastomers formed from divinyl compounds, particularly siloxane polymers and organic materials with at least two free vinyl groups, reacting with Si—H linkages on a polysiloxane backbone. Non-limiting examples of emulsifying crosslinked organopolysiloxane suitable for use in the liquid cosmetic compositions of the present invention are described in U.S. Pat. No. 5,412,004 (Tachibana et. al., issued May 2, 1995); U.S. Pat. No. 5,837,793 (Harashima et. al., issued Nov. 17, 1998); 6,103,250 (Brieva et al., issued Aug. 15, 2000); U.S. Pat. No. 5,919,437 (Lee et al., issued Jul. 6, 1999) and U.S. Pat. No. 5,811,487 (Schulz, Jr. et. al., issued Sep. 22, 1998), which descriptions are incorporated herein by reference.

An example of a specific silicone gel material containing an emulsifying silicone elastomer for use herein is the dimethicone (and) dimethicone copolyol crosspolymer available from Shin Etsu under the tradename KSG-21, and DC-9010 and DC-9011 available from Dow Corning.

Non-limiting examples of specific silicone elastomers, silicone elastomer gels, silicone rubber, and silicone elastomer powders, suitable for use in the liquid cosmetic compositions of the present invention include dimethicone crosspolymer, dimethicone (and) dimethicone copolyol crosspolymer, dimethicone/vinyl dimethicone crosspolymer, cyclomethicone (and) vinyl dimethicone/methicone crosspolymer, crosslinked stearyl methyl dimethyl siloxane copolymer, cetearyl dimethicone/vinyl dimethicone crosspolymer, cyclomethicone [and] dimethicone crosspolymers, and combinations thereof. Such dimethicone/vinyl dimethicone crosspolymers and cyclomethicone (and) dimethicone crosspolymers are supplied by a variety of suppliers including Dow Corning (DC 9506), General Electric (SFE-167, SFE-168, SFE-839), Shin Etsu (KSG-15 [cyclopentasiloxane (and) dimethicone/vinyl crosspolymer], KSG-16 [dimethicone (and) dimethicone/vinyl dimethicone crosspolymer], KSG-17, KSG-18 [phenyltrimethicone (and) dimethicone/phenyl vinyl dimethicone crosspolymer, KSG-20, KSG-21 [dimethicone (and) dimethicone copolyol crossspolymer]), and Grant Industries (Gransil™ line of materials, such as Gransil SR-SYC). Such cyclomethicone [and] dimethicone crosspolymers are supplied by a variety of suppliers including Dow Corning DC 9040. Other silicone elastomers supplied by Dow Corning include DC 9010, DC 9011, DC 9041, DC 9070, DC 9090 and BY29-119.

Other suitable crosslinked organopolysiloxanes and processes for making them are described in U.S. Pat. No. 4,970,252 (Sakuta et al., issued Nov. 13, 1990); U.S. Pat. No. 5,760,116 (Kilgour et al., issued Jun. 2, 1998); and U.S. Pat. No. 5,654,362 (Schulz, Jr. et al. issued Aug. 5, 1997), which descriptions are incorporated herein by reference. The silicone elastomers for use in the liquid cosmetic compositions of the present invention are preferably cured under anhydrous conditions or in an anhydrous environment. More preferably, the silicone elastomers have one or more moieties such as alkyl, phenyl, styryl, oxyalkylene, polyoxyalkylene, or combinations thereof.

It has been found that silicone elastomers in the form of powders or gels can be added to or formulated in the liquid cosmetic compositions of the present invention, while also maintaining Coverage and Wear Indices within the ranges as defined herein.

### Product Performance

The liquid cosmetic compositions of the present invention can also be characterized as liquid extended-wear formulations that provide or otherwise deliver functional characteristics such as non-tacky application and improved coverage over large areas of the skin. These functional characteristics include Average Coverage Index (ACI), Average Sweat Wear Index (ASWI), and Average Tackiness Index (ATI), all of which are described in detail hereinafter. All measurements are made under ambient condition (25° C., 50%RH).

The liquid cosmetic compositions of the present invention are in liquid form under ambient conditions and have a viscosity of less than 100,000 centistokes, preferably from about 1,000 to about 50,000 centistokes, more preferably from about 4,000 to about 30,000 centistokes, as measured by a viscometer, Brookfield DV-II+, at 1 rpm and 25° C.

1. Coverage Index

The liquid cosmetic compositions of the present invention preferably provide an Average Coverage Index (ACI) of at least about 80%, preferably at least 85%, wherein the Average Coverage Index is determined by the test methodology described hereinafter.

It has been found that the Coverage Index as determined by the test methodology described herein helps predict the effectiveness of a liquid cosmetic composition in covering or otherwise hiding skin discolorations or imperfections. Evaluation by this method includes analysis of an image of a product-treated artificial skin-like collagen surface. The image is analyzed for the average amount of light blocked by the product and a “Coverage Index” is calculated. The Coverage Index is thus a measure of the ability of a liquid cosmetic composition to effectively cover skin imperfections and discolorations.

The equipment and materials needed for the Coverage Index test methodology includes the following:

1) Transmission microscope (Zeiss SV-11 stereo microscope) equipped with Sony DXC-760MD Camera and reflected light source.

2) Computer image system with software for measuring image brightness.

3) A calibration disc covered by a black tape in one half while the other half is clear.

4) Collagen film such as VITRO-SKIN #1192 from IMS inc.

5) Circular collagen film holder with a snap ring. The inner diameter is 3.5 cm.

6) Constant humidity chamber adjusted to 95% relative humidity.

7) Analytical balance.

8) Ruler.

9) Utility knife.

The Coverage Index methodology then includes the following procedural steps or operations:

1) Prepare a sheet of collagen substrate by hydrating it in a 95% relative humidity chamber for at least two hours.

2) Remove the collagen sheet from the hydration box. Cut the sheet into 2.3×3.3 cm rectangle film using a ruler and a utility knife.

3) Immediately wrap the collagen film on the film holder. The top collagen surface should be flat and free of wrinkles.

4) Snap the collagen holder ring straight down into place.

5) Allow the collagen film to equilibrate at the ambient conditions for 24 hours.

6) Prepare a Control Product based on the composition below and make sure the product is homogeneous:

| Ingredient | Weight Percent |
|---|---|
| MQ Resin¹ | 15.00 |
| Silicone Gum² | 7.50 |
| Rutile TiO2 | 21.19 |
| Yellow Iron Oxide | 2.66 |
| Black Iron Oxide | 0.19 |
| Red Iron Oxide | 0.96 |
| Bentone Gel ISD³ | 31.50 |
| Propyl Paraban | 0.20 |
| Isododecane | Q.S. |
| Total | 100 |

¹MQ Resin (SR1000 - General Electric)
²Dimethicone gum - (25,000,000 cst) (SE30 -General Electric)
³Bentone Gel ISD - 10% Hectorite Clay, 3% propylene carbonate, 87% isododecane (VS-5 PC -Rheox)

7) Weigh 0.0100±0.0002 g of control product using a four-place analytical balance and immediately spread the product on the collagen film in circles to cover the entire collagen surface with a surface area of 9.6 cm², completing the application in 10 gentle strokes.

8) Allow the control to dry for 5 minutes before taking the coverage measurements.

9) Calibrate the imaging analysis system using the calibration disc.

10) Place a blank collagen film in the center of the light path on the microscope base. Adjust the focus and take an initial reading of the light brightness for the blank.

11) Move the blank collagen film slightly off-center and take readings of the light brightness from six random positions. The average light brightness reading from all seven positions is the Blank Mean.

12) Replace the blank with a collagen film with control sample in the center of the light path on the microscope base. Adjust focus and take an initial reading of the light brightness.

13) Move the control collagen film off-center and take light brightness readings of six random positions. The average light brightness reading from all seven positions is the Control Mean.

14) Calculate the control coverage reading based on the equation below:

Control Coverage=(Blank Mean−Control Mean)/Blank Mean

15) Weigh 0.0100±0.0002 g of a test product using a four-place analytical balance and immediately spread the product on the collagen film in circles to cover the entire collagen surface with a surface area of 9.6 cm², completing the application in 10 gentle strokes.

16) Allow the test product to dry for 5 minutes before taking the coverage measurements.

17) Place a blank collagen film in the center of the light path on the microscope base. Adjust the focus and take an initial reading of the light brightness for the control.

18) Move the blank collagen film slightly off-center and take readings of the light brightness from six random positions. The average light brightness reading from all seven positions is the Blank Mean.

19) Replace the blank with a Sample collagen film in the center of the light path on the microscope base. Adjust focus and take an initial reading of the light brightness.

20) Move the Sample collagen film off-center and take light brightness readings of six random positions. The average light brightness reading from all seven positions is the Sample Mean.

21) Calculate the Sample coverage reading based on the equation below:

Sample Coverage=(Blank Mean−Sample Mean)/Blank Mean

22) The Coverage Index of the test product is calculated based on the equation below:

Coverage Index=Sample Coverage/Control Coverage*100%

Steps (7) through (22) are repeated eight times for each liquid cosmetic composition, and an average Coverage Index value obtained from the eight Coverage Index values thus obtained. The liquid cosmetic compositions of the present invention preferably provide an Average Coverage Index (ACI) of at least about 80%, preferably at least about 85%.

2. Dry Wear Index

The liquid cosmetic compositions of the present invention preferably provide an Average Wear Index (AWI) of at least about 60%, more preferably at least about 65%, even more preferably from about 70% to 100%, wherein the Average Wear Index is determined by the test methodology described hereinafter (Dry Blot and Rub Test Method). The methodology for determining the Average Wear Index provides a means for determining or predicting the ability of a liquid cosmetic composition to resist color transfer upon contact with objects.

The Average Wear Index is an indirect measure of the extended wear properties of the liquid cosmetic compositions of the present invention, and is determined by the following Dry Blot and Rub Test Methodology. Equipment and material for use in the test method include the following:

1) Transmission microscope (Zeiss SV-11 stereo microscope) equipped with Sony DXC-760MD Camera and reflected light source.

2) Computer image system with software for measuring image brightness.

3) A calibration disc covered by a black tape in one half while the other half is clear.

4) Collagen film such as VITRO-SKIN#1192 from IMS inc.

5) Collagen film holder with a snap ring.

6) Constant humidity chamber adjusted to 95% relative humidity.

7) Analytical balance.

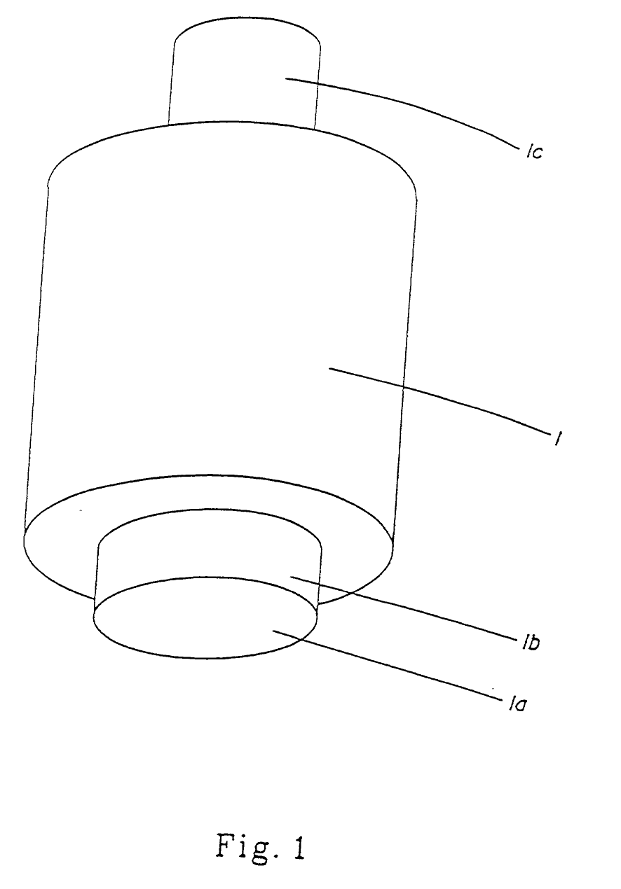

8) 2 kg weight attached with Aluminum disc illustrated in FIG. 1.

9) Ruler.

10) Utility knife

11) Bounty Paper towel

12) Single-sided adhesive tape.

The Average Wear Index for the liquid cosmetic compositions of the present invention are then determined by the following procedural steps or operations:

1) Prepare a sheet of collagen substrate by hydrating it in a 95% relative humidity chamber for at least two hours.

2) Remove the collagen sheet from the hydration box. Cut the sheet into 2.3×3.3 cm rectangle film using a ruler and a utility knife.

3) Immediately wrap the collagen film on the film holder. The top collagen surface should be flat and free of wrinkles.

4) Snap the collagen holder ring straight down into place.

5) Allow the collagen film to equilibrate at the ambient conditions for 24 hours.

6) Calibrate the imaging analysis system using the calibration disc.

7) Weigh 0.0100±0.0002 g of test product using a four-place analytical balance and immediately spread the product on the collagen film in circles to cover the entire collagen surface, completing the application in 10 gentle strokes.

8) Allow the sample to dry for 5 minutes before taking the coverage measurements.

9) Place a blank collagen film in the center of the light path on the microscope base. Adjust the focus and take an initial reading of the light brightness for the blank. Move the blank collagen film slightly off-center and take the light brightness readings from six random positions. The average light brightness reading from all seven positions is the Blank Mean.

10) Replace the blank with a sample collagen film in the center of the light path on the microscope base. Adjust the focus and take readings of the light brightness from all six remaining positions (similar to the blank). The average light brightness reading is the Sample Mean.

11) Calculate the initial coverage index based on the equation below:

Initial Coverage=(Blank Mean−Sample Mean)/Blank Mean

12) Tape down a sheet of Bounty paper towel on a flat desk surface using a single-sided adhesive tape around the edge area.

13) Allow the collagen sample to dry at ambient for 30 minutes after the initial product application in step (7).

14) Gently place the collagen sample on the paper towel with the surface with sample products touching the paper towel.

15) Set the 2 kg weight with the aluminum block attached in FIG. 1 on top of the collage film. It is important to position the weight gently so that excess force beyond 2 kg is not applied.

16) Grasping the collagen holder and carefully rotate the disk through 360° while maintaining the 2 kg force on the film. Do not lift or press the weight into the film during the rotation to the weight.

The entire 360° rotation should be completed within a time interval between 3 to 5 seconds.

17) Lift the weight straight up off the film surface.

18) Measure the coverage of the remaining product film through steps (9) and (10) for both blank and the remaining sample.

19) Calculate the Remaining Coverage based on the equation below:

Remaining Coverage=(Blank Mean−Sample Mean)/Blank Mean

20) The Dry Wear Index is calculated based on the equation below:

Dry Wear Index=Remaining Coverage/Initial Coverage*100%

Steps (7) through (20) are repeated eight times for each liquid cosmetic composition, and an Average Dry Wear Index (ADWI) value obtained from the eight Wear Index values thus obtained.

3. Sweat Wear Index

The liquid cosmetic compositions of the present invention preferably provide an Average Sweat Wear Index (ASWI) of at least about 60%, more preferably at least about 65%, even more preferably at least 70%, wherein the Average Wear Index is determined by the test methodology described hereinafter (Sweat Blot and Rub Test Method). The methodology for determining the Average Sweat Wear Index provides a means for determining or predicting the ability of a liquid cosmetic composition to resist color transfer under sweaty conditions upon contact with objects.

The Average Sweat Wear Index is thus an indirect measure of the extended wear properties of the liquid cosmetic compositions of the present invention, and is determined by the following Sweat Blot and Rub Test Methodology. Equipment and material for use in the test method include the following:

1) Transmission microscope (Zeiss SV-11 stereo microscope) equipped with Sony DXC-760MD Camera and reflected light source.

2) Computer image system with software for measuring image brightness.

3) A calibration disc covered by a black tape in one half while the other half is clear.

4) Collagen film such as VITRO-SKIN#1192 from IMS inc.

5) Collagen film holder with a snap ring.

6) Constant humidity chamber adjusted to 95% relative humidity.

7) Analytical balance.

8) 2 kg weight attached with Aluminum disc illustrated in FIG. 1.

9) Ruler.

10) Utility knife

11) Bounty Paper towel

12) Single-sided adhesive tape.

The Average Sweat Wear Index for the liquid cosmetic compositions of the present invention are then determined by the following procedural steps or operations:

1) Prepare a sheet of collagen substrate by hydrating it in a 95% relative humidity chamber for at east two hours.

2) Remove the collagen sheet from the hydration box. Cut the sheet into 2.3×3.3 cm rectangle film using a ruler and a utility knife.

3) Immediately wrap the collagen film on the bottom piece of the holder. The top collagen surface should be flat and free of wrinkles.

4) Snap the collagen holder ring straight down into place.

5) Allow the collagen film to equilibrate at the ambient conditions for 24 hours.

6) Weigh 0.0100±0.0002 g of test product using a four-place analytical balance and immediately spread the product on the collagen film in circles to cover the entire collagen surface, completing the application in 10 gentle strokes.

7) Allow the sample to dry for 5 minutes before taking the coverage measurements.

8) Calibrate the imaging analysis system using the calibration disc.

9) Place a blank collagen film in the center of the light path on the microscope base. Adjust the focus and take an initial reading of the light brightness for the blank. Move the blank collagen film slightly off-center and place into one of the six rosette petals and take the second reading of the light brightness. Continue the process and collect the data from all remaining positions. The average light brightness reading from all positions is the Blank Mean.

10) Replace the blank with a sample collagen film in the center of the light path on the microscope base. Adjust the focus and take readings of the light brightness from all six remaining positions (similar to the control). The average light brightness reading is the Sample Mean. 11) Calculate the initial coverage index based on the equation below:

Initial Coverage=(Blank Mean−Sample Mean)/Blank Mean

12) Tape down a sheet of Bounty paper towel on a flat desk surface using a single-sided adhesive tape around the edge area.

13) Allow the collagen sample to dry at ambient for 30 minutes after the initial product application in step (6).

14) Prepare an artificial sweat solution based on the composition below:

| Ingredient | Weight Percent |
|---|---|
| NaCl | 0.3214 |
| Na₂CO₃ | 0.1428 |
| Lactic Acid | 0.1802 |
| KOH | 0.0281 |
| Urea | 0.0200 |
| NH₄OH | 0.0140 |
| Pyruvic Acid | 0.0008 |
| Glucose | 0.0004 |
| Water | Q.S. |
| Total | 100% |

15) Using a pipet, drop 0.1 grams of above artificial sweat solution onto the dried film.

16) Gently distribute the sweat solution evenly over the film surface using an index finger in 10 circular motions.

17) Allow the sweat to remain on the film undisturbed for 10 minutes.

18) Place the collagen sample on the paper towel with the product surface touching the paper towel.

19) Set the 2 kg weight with the aluminum block attached in FIG. 1 on top of the collage film. It is important to position the weight gently so that excess force beyond 2 kg is not applied.

20) Grasping the collagen holder and carefully rotate the disk through 3600 while maintaining the 2 kg force on the film. Do not lift or press the weight into the film during the rotation to the weight. The entire 360m° rotation should be completed within a time interval between 3 to 5 seconds.

21) Lift the weight straight up off the film surface.

22) Measure the coverage of the remaining product film through step (9) and (10) for both blank and the remaining sample.

23) Calculate the Remaining Coverage based on the equation below:

Remaining Coverage=[(Blank Mean−Sample Mean)/Blank Mean]

24) The Sweat Wear Index is calculated based on the equation below:

Sweat Wear Index=Remaining Coverage/Initial Coverage*100%

Steps (6) through (24) are repeated eight times for each liquid cosmetic composition, and an Average Sweat Wear Index (ASWI) value is determined from the eight Sweat Wear Index values thus obtained.

4. Oil Wear Index

The liquid cosmetic compositions of the present invention preferably provide an Average Oil Wear Index (AOWI) of at least about 60%, more preferably at least about 65%, even more preferably from about 70% to 100%, wherein the Average Oil Wear Index is determined by the test methodology described hereinafter (Oil Blot and Rub Test Method). The methodology for determining the Average Oil Wear Index provides a means for determining or predicting the ability of a liquid cosmetic composition to resist color transfer from an oily surface, e.g., oily skin, upon contact with objects.

The Average Sweat Wear Index is thus an indirect measure of the extended wear properties of the liquid cosmetic compositions of the present invention, and is determined by the following Oil Blot and Rub Test Methodology. Equipment and material for use in the test method include the following:

1) Transmission microscope (Zeiss SV-11 stereo microscope) equipped with Sony DXC-760MD Camera and reflected light source.

2) Computer image system with software for measuring image brightness.

3) A calibration disc covered by a black tape in one half while the other half is clear.

4) Collagen film such as VITRO-SKIN#1192 from IMS inc.

5) Collagen film holder with a snap ring.

6) Constant humidity chamber adjusted to 95% relative humidity.

7) Analytical balance.

8) 2 kg weight attached with Aluminum disc illustrated in FIG. 1.

9) Ruler.

10) Utility knife

11) Bounty Paper towel

12) Single-sided adhesive tape.

The Average Oil Wear Index for the liquid cosmetic compositions of the present invention are then determined by the following procedural steps or operations:

1) Prepare a sheet of collagen substrate by hydrating it in a 95% relative humidity chamber for at least two hours.

2) Remove the collagen sheet from the hydration box. Cut the sheet into 2.3×3.3 cm rectangle film using a ruler and a utility knife.

3) Immediately wrap the collagen film on the bottom piece of the holder. The top collagen surface should be flat and free of wrinkles.

4) Snap the collagen holder ring straight down into place.

5) Allow the collagen film to equilibrate at the ambient conditions for 24 hours.

6) Weigh 0.0100±0.0002 g of test product using a four-place analytical balance and immediately spread the product on the collagen film in circles to cover the entire collagen surface, completing the application in 10 gentle strokes.

7) Allow the sample to dry for 5 minutes before taking the coverage measurements.

8) Calibrate the imaging analysis system using the calibration disc.

9) Place a blank collagen film in the center of the light path on the microscope base. Adjust the focus and take an initial reading of the light brightness for the control. Move the blank collagen film slightly off-center and take the light brightness readings at six random positions. The average light brightness reading from all seven positions is the Blank Mean.

10) Replace the blank with a sample collagen film in the center of the light path on the microscope base. Adjust the focus and take readings of the light brightness from six random positions (similar to the blank). The average light brightness reading is the Sample Mean.

11) Calculate the initial coverage based on the equation below:

Initial Coverage=(Blank Mean−Sample Mean)/Blank Mean

12) Tape down a sheet of Bounty paper towel on a flat desk surface using a single-sided adhesive tape around the edge area.

13) Allow the collagen sample to dry at ambient for 30 minutes after the initial product application in step (6).

14) Using a pipet, drop 0.1 grams of olive oil onto the dried film.

15) Gently distribute the olive oil evenly over the film surface using an index finger in 10 circular motions.

16) Allow the oil to remain on the film undisturbed for 10 minutes.

17) Place the collagen sample on the paper towel with the product surface touching the paper towel.

18) Set the 2 kg weight with the aluminum block attached in FIG. 1 on top of the collage film. It is important to position the weight gently so that excess force beyond 2 kg is not applied.

19) Grasping the collagen holder and carefully rotate the disk through 360° while maintaining the 2 kg force on the film. Do not lift or press the weight into the film during the rotation to the weight.

The entire 360° rotation should be completed within a time interval between 3 to 5 seconds.

20) Lift the weight straight up off the film surface.

21) Measure the coverage of the remaining product film through step (9) and (10) for both blank and the remaining sample.

22) Calculate the Remaining Coverage based on the equation below:

Remaining Coverage=(Blank Mean−Sample Mean)/Blank Mean

23) The Oil Wear Index is calculated based on the equation below:

Oil Wear Index=Remaining Coverage/Initial Coverage*100%

Steps (6) through (23) are repeated eight times for each liquid cosmetic composition, and an Average Oil Wear Index (ASWI) value is determined from the eight Sweat Wear Index values thus obtained.

5. Film Flexibility

The liquid cosmetic compositions of the present invention preferably provide an Average Percent Weight Loss as determined by the following Film Flexibility Method, of less than about 30%, preferably less than about 15%, more preferably from zero to about 10%. The Film Flexibility Method provides a means for determining or predicting the ability of a liquid cosmetic composition to form a firm but flexible film on the skin that is sufficiently hard to provide transfer resistant but sufficiently flexible to stretch and move with body to thus provide extended wear performance.

The Average Percent Weight Loss as determined by the Film Flexibility Method is thus an indirect measure of the ability of the liquid cosmetic composition to form a firm but flexible film on the skin. The test was developed to essentially characterize the flexibility of the film formed from the topical application of the liquid cosmetic composition. The optimum test conditions to reliabily correlate this test to the physical characteristics of the composition requires that the film be dry. In this context, the term “dry” means that at least 90% of the volatile carrier of the liquid cosmetic composition tested has evaporated. This test of the film formed from the composition of the present invention is as follows:

Flexibility is measured by the latex stretch test. This test predicts the ability of the applied cosmetic film to resist flaking or peeling after application by movement during normal activities. The latex stretch test method is as follows:

Equipment:

1. Ansell Edmont Industrial technicians unlined gloves (12″ length, 17 mil) USDA Accepted#390, Size 9;

2. A disposable lip brush such as those available from La Femme Cosmetics, Inc. of L.A.

3. Analytical balance (4 decimal places);

4. Ruler; and

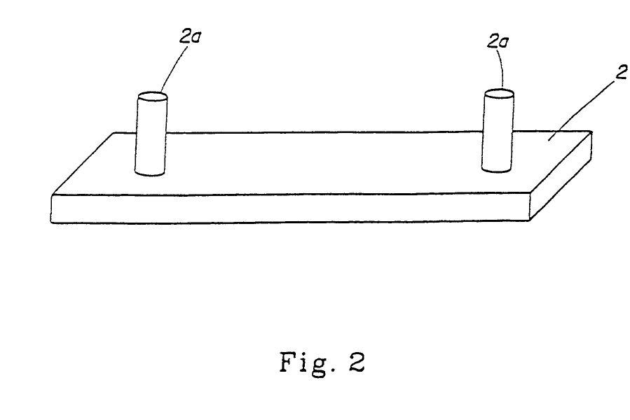

5. An apparatus as illustrated in FIG. 2. Said apparatus can be constructed from Lucite sheet and rod stock wherein posts 2a are approximately 6 inches apart.

Procedure:

(1) Cut a 1 inch wide band from the wrist area of the glove, avoiding the ribbing and thumb.

(2) Mark off a 1×1 inch block in the center of the band, avoiding the embossed number.

(3) Weigh and record the weight of the latex band; hereinafter referred to as A.

(4) Determine the initial weight of the cosmetic to be applied to the band in order to produce a dried film weighing 20 mg. This is determined by dividing 20 mg by the weight percent of non-volatile material present in the cosmetic. For example, 50 mg of a cosmetic with 40% non-volatile content must be applied to the band in order to yield a 20 mg dried film.

(5) Using a disposable lip brush, evenly apply the amount of cosmetic determined in step (4) over the 1×1 inch area of the band as marked in step (2).

(6) Immediately weigh and record the combined weight of the latex band and applied cosmetic. The wet film weight is calculated by subtracting A from the combined weight of the latex band and applied cosmetic.

(7) Allow the sample on the latex band from step (6) to sit at ambient room conditions for 24 hours.

(8) Weigh and record the combined weight of the latex band A and the applied cosmetic film; hereinafter referred to as B. Subtract A from B to determine the dried film weight C. This weight should be 20±2 mg.

(9) Stretch the band just enough to slip over the posts (2a) of apparatus (1) of FIG. 2. Gently manipulate the latex band on the posts so that the stretched film length is 1.75 inches.

(10) Upon observing loosened film pieces on the latex band, remove the film pieces from the latex band by vigorously wiping a disposable lip brush across the surface of the film.

(11) Carefully remove the latex band from the posts (2a) allowing it to returns to its approximate original shape.

(12) Record the weight of the latex band (with the remaining cosmetic); herein referred to as D.

(13) Calculate the percent weight loss of the cosmetic film using the following equation:

Percent Weight Loss (PWL)=[(D—A)÷(B—A)]×100

Steps (1) through (13) are repeated four times for each liquid cosmetic composition, and an Average Percent Weight Loss (APWL) value is determined from the four Percent Water Loss values thus obtained. Lower APWL values correspond to flexible films having desirable adhesive and cohesive balance of the film.

6. Tackiness Index

The liquid cosmetic compositions of the present invention preferably provide an Average Tackiness Index (ATI) of less than about 25%, preferably less than about 10%, even more preferably less than about 5%, wherein the Average Tackiness Index determined by the test methodology described hereinafter.

It has been found that the compositions of the present invention can provide extend wear and improved coverage, while also providing smooth, non-tacky cosmetics during and after application to the skin. The Average Tackiness Index is thus a measure of the extent to which a liquid cosmetic composition forms a smooth, non-tacky film on an applied surface. Lower ATI values mean that the tested composition results in a less tacky or sticky film on the skin relative to other compositions having higher ATI values. Equipment and material used in the test methodology include:

1) Instron Instrument (Model 5542, Instron Corporation)

2) Computer interfaced to the Instron with software for measuring peak force.

3) Analytical balance.

4) Ruler.

5) Utility knife

6) Silicone Rubber Substate

7) Double-sided adhesive tape.

Procedure:

1) Prepare a sheet of Silicone Rubber substrate and clean the substrate with isopropanol.

2) Cut the Silicone Rubber substrate into strips of one-inch width.

3) Cut the Silicone Rubber strips into smaller pads, half of the substrate pads are in square pieces with 1×1 inch and the other half of the pads are in rectangle pieces with 1×2 inch.

4) Place a metal cube on the base of the instron measurement plate.

5) Attach measurement probe to the load cell and make sure it is balanced.

6) Attach a blank squire pad (1×1 inch) to the base metal cube using a double-sided tape.

7) Attach a blank rectangle pad (1×2 inch) to the top probe using a double-sided tape. Adjust the pad position for maximum overlap between the top and bottom pads.

8) Calibrate the system and make fine adjustment of the load cell position to provide a load pressure of 100gs-150gf during the measurement.

9) Apply 0.04 ml of Glycerin control on the bottom pad (1×1 inch) and gently spread the product to cover the surface in 10 circular motions.

10) Wait 1 min before taking the tackiness measurement.

11) Take an initial tackiness reading. Repeat the tackiness measurements for a total of 10 times at 12-second intervals. The total summation of peak forces from these 10 tackiness measurements is assigned as the tackiness value of the Glycerin control.

12) Repeat steps 6-8 and place the new testing pads.

13) Apply 0.04 ml of test product on the bottom pad (1×1 inch) and gently spread the product to cover the surface in 10 circular motions.

14) Wait 1 min before taking the tackiness measurement.

15) Take an initial tackiness reading. Repeat the tackiness measurements 10 times at 12-second intervals. The total summation of peak forces from these 10 tackiness measurements is assigned as the tackiness value of the test product.

16) The tackiness index of the test product is calculated based on the following equation:

Tackiness Index=Tackiness of test product/Tackiness of Glycerin control*100%

Steps (1) through (16) are repeated eight times for each liquid cosmetic composition, and an Average Tackiness Index (ATI) value is determined from the eight Tackiness Index values thus obtained. Lower ATI values correspond to less sticky or tacky films.

### Optional Ingredients

The liquid cosmetic compositions of the present invention may further comprise other optional components which may modify the physical, chemical, cosmetic or aesthetic characteristics of the compositions or serve as additional “active” components when deposited on the skin. The compositions may also further comprise optional inert ingredients. Many such optional ingredients are known for use in personal care compositions, and may also be used in the liquid cosmetic compositions herein, provided that such optional materials are compatible with the essential materials described herein, or do not otherwise unduly impair product performance.

Such optional ingredients are generally those materials approved for use in cosmetics and that are described in reference books such as the CTFA Cosmetic Ingredient Handbook, Second Edition, The Cosmetic, Toiletries, and Fragrance Association, Inc. 1988, 1992. Non limiting examples of such optional ingredients include preservatives, deodorants, antimicrobials, fragrances, deodorant perfumes, coloring agents or dyes, thickeners, sunscreens, suspending agents, pH modifiers, surfactants, co-solvents, emollients, pharmaceutical actives, vitamins, and combinations thereof.

### Method of Use

The liquid cosmetic compositions of the present invention are applied topically to the desired area of the skin in an amount sufficient to cover or hide skin imperfections or discolorations. The compositions are especially effective when applied topically to areas of the skin discolored by visible varicose or spider veins, preferably to those skin discolorations on the arms and legs, especially the legs. The liquid composition may be applied to the desired area as needed, preferably once or twice daily, more preferably once daily, and then preferably allowed to dry before subjecting to contact such as with clothing or other objects. The composition is preferably applied to the desired area of the skin that is dry or has been dried prior to application.

### Method of Manufacture

The liquid cosmetic compositions of the present invention may be prepared by any known or otherwise effective technique, suitable for making and formulating a liquid topical suspension or dispersion. Specific non limiting examples of such methods as they are applied to the compositions of the present invention are described in the examples set forth below.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, unless otherwise specified.

The compositions described below in Examples A-I are liquid cosmetic liquid compositions that provide Wear and Coverage Indices within the ranges defined herein. Each of the compositions is packaged in a suitable container or application system, then applied topically to the skin to cover or hide skin imperfections or discolorations, including skin discoloration from varicose veins and spider veins, especially when such discolorations are on the legs. The compositions are more wear resistant even when applied over areas of the skin that contact clothing.

Each of the compositions described below (Examples A-J) is formulated by combining the MQ resin and isododecane in a 3:1 weight ratio with agitation using a propeller mixer until a uniform mixture results. Dimethicone gum and isododecane are likewise combined using a propeller mixer until a uniform mixture results, the weight ratio of the gum to isododecane in the premix being 3:2. The dimethicone gum premix, the Bentone Gel, and 50% of the MQ resin premix are combined with agitation to form a uniform mixture. The remaining MQ resin premix is then added to a separate propeller mixer and agitated to a uniform mixture before adding solid pigments, silicone elastomer (if any) and propylparaben, and then homogenizing the resulting mixture at 2000 rpm until all ingredients are fully dispersed. The resulting dispersion is then combined with the dimethicone gum premix with agitation in a propeller mixer until a uniform fluid results. The uniform fluid is transferred to and sealed within individual packages.

Each of the compositions is also prepared by first forming the dimethicone/isododecane premix as described above, and then adding all of the remaining ingredients, including dry powders such as the solid pigment, and then mixing the combined ingredients to incorporate the dry powders into the resulting composition. The resulting composition is then homogenized as described above until the dry powders are fully dispersed, and then transferred to and sealed within individual packages.

| Ingredients | A | B | C | D | E | F | G | H | I | J |
|---|---|---|---|---|---|---|---|---|---|---|
| MQ Resin¹ | 15.00 | 15.00 | 15.00 | 15.00 | 15.00 | 15.00 | 15.00 | 15.00 | 15.00 | 15.00 |
| Dimethicome Gum² | 7.50 | 7.50 | 7.50 | 7.50 | 7.50 | 5.00 | 7.50 | 7.50 | 5.00 | 6.75 |
| Bentone Gel³ | 27.39 | 27.39 | 34.20 | 31.50 | 31.50 | 30.00 | 30.00 | 20.00 | 30.00 | 25.00 |
| Total Solid Pigments | 25.00 | 15.00 | 15.00 | 20.00 | 20.00 | 20.00 | 20.00 | 20.00 | 20.00 | 20.00 |
| Anatasa TiO2 | 15.99 | 9.59 | 9.59 | | | | | | | |
| Rutile TiO2 | | | | 16.95 | 10.69 | 16.95 | 16.95 | 16.95 | 16.95 | 16.95 |
| Yellow Iron Oxide | 5.93 | 3.56 | 3.56 | 2.13 | 6.32 | 2.13 | 2.13 | 2.13 | 2.13 | 2.13 |
| Red Iron Oxide | 2.61 | 1.57 | 1.57 | 0.77 | 2.21 | 0.77 | 0.77 | 0.77 | 0.77 | 0.77 |
| Black Iron Oxide | 0.48 | 0.29 | 0.29 | 0.15 | 0.78 | 0.15 | 0.15 | 0.15 | 0.15 | 0.15 |
| Propylparaben | 0.20 | 0.20 | 0.20 | 0.20 | 0.20 | 0.20 | 0.20 | 0.20 | 0.20 | 0.20 |
| Isododecane | 24.91 | 34.91 | 28.10 | 25.79 | 25.80 | 28.79 | 24.29 | 34.29 | 26.79 | 31.04 |
| Elastomer | 0.00 | 0.00 | 0.00 | 0.00 | 0.00 | 1.00 | 3.00 | 3.00 | 3.00 | 2.00 |

¹MQ Resin (SR1000 -General Electric)
²Dimethicone gum - 25,000,000 cst, (SE30 -General Electric)
³Bentone Gel - 10% Hectorite Clay, 3% propylene carbonate, 87% isododecane (VS-S PC -Rheox)
⁴Silicone elastomer- dimethicone crosspolymers (Dow Corning DC9506)

What is claimed is:

1. Liquid cosmetic compositions for hiding or covering skin imperfections or discolorations, said compositions being in liquid form and characterized by an average Coverage Index of at least about 80%, an average Sweat Wear Index of at least about 60%, and a Tackiness Index of less than about 25%.

2. A liquid cosmetic composition according to claim 1, wherein the average Coverage Index is at least about 85%.

3. A liquid cosmetic composition according to claim 1, wherein the average Sweat Wear Index is at least about 70%.

4. A liquid cosmetic composition according to claim 1 wherein the Tackiness Index is less than about 10%.

5. A liquid cosmetic composition according to claim 1, wherein the composition further comprises:
(A) an organosiloxane resin and a fluid diorganopolysiloxane polymer in a weight ratio of the resin to polymer of from about 1:2 to about 10:1;
(B) from about 10% to about 90% by weight of a volatile carrier liquid;
(C) from about 15% to about 50% by weight of a solid pigment such that the weight ratio of the fluid diorganopolysiloxane polymer and organosiloxane resin to the solid pigment is from about 0.50 to about 2.5; and
(D) from about 0.5% to about 15% by weight of an inorganic thickening solid.

6. A liquid cosmetic composition according to claim 5, wherein the composition is anhydrous and contains less than 10% by weight of water.

7. A liquid cosmetic composition according to claim 5, wherein the average Tackiness Index is less than about 10%.

8. A liquid cosmetic composition according to claim 5, wherein the composition further comprises a silicone elastomer.

9. A liquid cosmetic composition according to claim 5, said composition containing from about 1% to about 5% by weight of an inorganic thickening solid in the form of a gel material comprising quaternium-18 hectorite, isododecane, and propylene carbonate.

10. A liquid cosmetic composition according to claim 1, wherein the composition further comprises:
(A) an organosiloxane resin and a fluid diorganopolysiloxane polymer in a weight ratio of the resin to polymer of from about 1:2 to about 10:1;
(B) from about 10% to about 90% by weight of a volatile carrier liquid;
(C) from about 15% to about 50% by weight of a solid pigment such that the weight ratio of the fluid diorganopolysiloxane polymer and organosiloxane resin to the solid pigment is from about 0.50 to about 2.5; and
(D) from about 0.1% to about 20% by weight of a silicone elastomer.

11. A liquid cosmetic composition according to claim 10, wherein the composition is anhydrous and contains less than 10% by weight of water.

12. A liquid cosmetic composition according to claim 10, wherein the average Tackiness Index is less than about 10%.

13. A liquid cosmetic composition according to claim 10, wherein the silicone elastomer is an emulsifying silicone elastomer.

14. A liquid cosmetic composition according to claim 10, wherein the silicone elastomer is a non-emulsifying silicone elastomer.

15. A liquid cosmetic composition for hiding or covering skin imperfections or discolorations, comprising:
(A) an organosiloxane resin and a fluid diorganopolysiloxane polymer in a weight ratio of the resin to polymer of from about 1:2 to about 10:1;
(B) from about 10% to about 90% by weight of a volatile carrier liquid;
(C) from about 15% to about 50% by weight of a solid pigment such that the weight ratio of the fluid diorganopolysiloxane polymer and organosiloxane resin to the solid pigment is from about 0.50 to about 2.5; and
(D) from about 0.5% to about 15% by weight of an inorganic thickening solid.

16. A liquid cosmetic composition according to claim 15, wherein the viscosity of the composition ranges from about 100 centistoke to about 100,000 centistoke as measured at 25° C.

17. A liquid cosmetic composition according to claim 15, wherein the weight ratio of the organosiloxane resin to the fluid diorganopolysiloxane polymer is from 1:1 to about 3:1.

18. A liquid cosmetic composition according to claim 15, wherein the fluid diorganopolysiloxane polymer and the organsiloxane resin together represent from about 10% to about 40% by weight of the composition.

19. A liquid cosmetic composition according to claim 15, wherein the fluid diorganopolysiloxane polymer represents from about 3% to about 15% by weight of the composition, has a viscosity of from about 100,000 centistokes to about 25 million centistokes as measured at 25° C., and comprises repeating units corresponding to the formula R₂SiO wherein the R₂ moieties are monvalent hydrocarbon radicals containing from about 1 to about 6 carbon atoms.

20. A liquid cosmetic composition according to claim 15, wherein the organosiloxane resin has a viscosity of from about 100,000 centistokes to about 25,000,000 centistokes as measured at 25° C. and contains functional groups selected from the group consisting of R₃SiO_{1/2} (M units), R₂SiO (D units), RSiO_{3/2} (T units), SiO₂ (Q units), and combinations thereof, the ratios of the groups to one another within the resin being such that the resin conforms to the formula RₙSiO_{(4-n)/2} where n is a value between 1.0 and 1.50 and R is a methyl group.

21. A liquid cosmetic composition according to claim 15, wherein the organosiloxane resin is an MQ resin in which the ratio of the M to Q functional units is from about 0.5 to about 1.5.

22. A liquid cosmetic composition according to claim 15, wherein the volatile carrier liquids comprise isododecane.

23. A liquid cosmetic composition according to claim 15, wherein the composition comprises from about 15% to about 30% by weight of the solid pigment and wherein the weight ratio of the fluid diorganopolysiloxane polymer and organosiloxane resin to the solid pigment is from about 0.5 to about 1.5.

24. A liquid cosmetic composition according to claim 15, wherein the inorganic thickening solids are selected from the group consisting of inorganic clay, silica, and mixtures thereof.

25. A liquid cosmetic composition according to claim 15, wherein the inorganic thickening solids comprise hydrophobically treated hectorite.

26. A liquid cosmetic composition according to claim 15, wherein the inorganic thickening solid comprise quaternium-18 hectorite.

27. A liquid cosmetic composition according to claim 15, wherein the inorganic thickening solid is formulated into the composition in the form of a gel material comprising quaternium-18 hectorite, isododecane, and propylene carbonate.

28. A liquid cosmetic composition according to claim 15, wherein the composition further comprises from about 0.1% to about 20% by weight of a silicone elastomer.

29. A liquid cosmetic composition according to claim 15, wherein the composition is anhydrous and contains less than 10% by weight of water.

30. A liquid cosmetic composition for hiding or covering skin imperfections or discolorations, comprising:
(A) an organosiloxane resin and a fluid diorganopolysiloxane polymer in a weight ratio of the resin to polymer of from about 1:2 to about 10: 1;
(B) from about 10% to about 90% by weight of a volatile carrier liquid;
(C) from about 15% to about 50% by weight of a solid pigment such that the weight ratio of the fluid diorganopolysiloxane polymer and organosiloxane resin to the solid pigment is from about 0.50 to about 2.5; and
(D) from about 0.1% to about 20% by weight of a silicone elastomer.

31. A liquid cosmetic composition according to claim 30, wherein the viscosity of the composition ranges from about 100 centistoke to about 100,000 centistoke as measured at 25° C.

32. A liquid cosmetic composition according to claim 30, wherein the weight ratio of the organosiloxane resin to the fluid diorganopolysiloxane polymer is from 1:1 to about 3:1.

33. A liquid cosmetic composition according to claim 30, wherein the fluid diorganopolysiloxane polymer and the organsiloxane resin together represent from about 10% to about 40% by weight of the composition.

34. A liquid cosmetic composition according to claim 30, wherein the fluid diorganopolysiloxane polymer represents from about 3% to about 15% by weight of the composition, has a viscosity of from about 100,000 centistokes to about 25 million centistokes as measured at 25° C., and comprises repeating units corresponding to the formula R₂SiO wherein the R₂ moieties are monvalent hydrocarbon radicals containing from about 1 to about 6 carbon atoms.

35. A liquid cosmetic composition according to claim 30, wherein the organosiloxane resin has a viscosity of from about 100,000 centistokes to about 25,000,000 centistokes as measured at 25° C. and contains functional groups selected from the group consisting of R₃SiO_{1/2} (M units), R₂SiO (D units), RSiO_{3/2} units), SiO₂ (Q units), and combinations thereof, the ratios of the groups to one another within the resin being such that the resin conforms to the formula RₙSiO_{(4-n)/2} where n is a value between 1.0 and 1.50 and R is a methyl group.

36. A liquid cosmetic composition according to claim 30, wherein the organosiloxane resin is an MQ resin in which the ratio of the M to Q functional units is from about 0.5 to about 1.5.

37. A liquid cosmetic composition according to claim 30, wherein the volatile carrier liquids comprise isododecane.

38. A liquid cosmetic composition according to claim 30, wherein the composition comprises from about 15% to about 30% by weight of the solid pigment and wherein the weight ratio of the fluid diorganopolysiloxane polymer and organosiloxane resin to the solid pigment is from about 0.5 to about 1.5.

39. A liquid cosmetic composition according to claim 30, wherein the silicone elastomer is an emulsifying silicone elastomer.

40. A liquid cosmetic composition according to claim 30, wherein the silicone elastomer is a non-emulsifying silicone elastomer.

41. A liquid cosmetic composition according to claim 30, wherein the silicone elastomer concentration ranges from about 0.1% to about 5%, by weight of the composition.

42. A liquid cosmetic composition according to claim 30, wherein the composition is anhydrous and contains less than 10% by weight of water.

43. A method of covering or hiding skin imperfections or discolorations, said method comprising application of the composition of claim 1 to skin imperfections or discolorations.

44. A method of covering or hiding skin imperfections or discolorations, said method comprising application of the composition of claim 1 to those areas of the skin discolored by visible varicose veins, spider veins, or combinations thereof.

45. A method of covering or hiding skin imperfections or discolorations, said method comprising application of the composition of claim 5 to skin imperfections or discolorations.

46. A method of covering or hiding skin imperfections or discolorations, said method comprising application of the composition of claim 5 to those areas of the skin discolored by visible varicose veins, spider veins, or combinations thereof.

47. A method of covering or hiding skin imperfections or discolorations, said method comprising application of the composition of claim 10 to skin imperfections or discolorations.

48. A method of covering or hiding skin imperfections or discolorations, said method comprising application of the composition of claim 10 to those areas of the skin discolored by visible varicose veins, spider veins, or combinations thereof.

49. A method of covering or hiding skin imperfections or discolorations, said method comprising application of the composition of claim 15 to skin imperfections or discolorations.

50. A method of covering or hiding skin imperfections or discolorations, said method comprising application of the composition of claim 15 to those areas of the skin discolored by visible varicose veins, spider veins, or combinations thereof.

51. A method of covering or hiding skin imperfections or discolorations, said method comprising application of the composition of claim 30 to skin imperfections or discolorations.

52. A method of covering or hiding skin imperfections or discolorations, said method comprising application of the composition of claim 30 to those areas of the skin discolored by visible varicose veins, spider veins, or combinations thereof.

53. A method of covering or hiding skin imperfections or discolorations, said method comprising application of the composition containing silicone resin to those areas of the skin discolored by visible varicose veins, spider veins, or combinations thereof.
